(19) United States
(12) Patent Application Publication
(10) Pub. No.: US 2007/0073225 A1
(43) Pub. Date: Mar. 29, 2007

(54) Retractable safety syringe

(76) Inventors: Shuei-Yuan Lee, Taipei City (TW); Pei-Hsin Pei, Taipei County (TW); Tu-Sing Hsueh, (US)

Correspondence Address: TROXELL LAW OFFICE PLLC, SUITE 1404, 5205 LEESBURG PIKE, FALLS CHURCH, VA, 22041, US

(21) Appl. No.: 11/181,735

(22) Filed: Jul. 15, 2005

(51) Int. Cl. A61M 5/00

(52) U.S. Cl. 604/110

(57) ABSTRACT

A retractable safety syringe includes primarily a barrel, a seat, and a push rod, wherein a tubular exit is located at an end of the barrel, a bore is located at an inner rim of bottom of exit, a thin breakage part is located at a periphery of the end, and a groove is located on a surface of barrel close to the end for latching and fixing a cover. A bolt is located at a top end of the push rod, so as to retract the push rod by locking the bolt of push rod into the bore at the inner rim of exit of barrel, after accomplishing an injection with the syringe, thereby breaking the breakage part at the periphery of barrel from a stretching force and retracting a needle into the barrel, in order to prevent the used syringe from being recycled.

### BACKGROUND OF THE INVENTION

(a) Field of the Invention

The present invention relates to a retractable safety syringe and more particularly to a syringe wherein a barrel will be broken and a needle will be retracted into the barrel by a retraction after taking an injection, so as to prevent the syringe from being recycled.

(b) Description of the Prior Art

For an ordinary syringe, as a needle is still fixed on a barrel after taking an injection, a common processing method is to separate the needle from the barrel, and destroy them separately. As the entire structure of syringe remains intact, there is a doubt that the syringe will be recycled by unworthy people during a process of destroying, thereby causing contamination and germ infection. Accordingly, some vendors have designed a variety of retractable syringes to retract needles into barrels to avoid reuse. As shown in FIG. 1, in the new patent 88211100, a needle 11 is provided with a latching structure 111, and a push rod 12 is provided with a hook 121, so as to retract the needle 11 by latching the hook 121 into the latching structure 111, after taking an injection. Referring to FIG. 2, the new patent 92201914 is provided with a retractable needle 21 and a latching part 221 in a front end of a push rod 22 corresponding to the needle 21, so as to retract the needle 21 by latching the latching part 221 onto the needle 21. Referring to FIG. 3, the new patent 91216494, a needle 31 is provided with a retracting mechanism 311, and a latching member 321 is installed in a front end of a push rod 32, such that the push rod 32 can retract the needle 31 by latching the latching member 321 onto the retracting mechanism 311, after taking an injection.

For the aforementioned new patents, the needle must be completely sealed with the barrel to prevent an inadvertent leaking of medicine, and the structure must be easily operated without exerting a large force when retracting the needle, independent of the method of connecting and retracting the push rod and the needle. Therefore, an issue of precision between these two compartments occurs, which induces a trouble to mass production, thereby causing a large imperfection rate, and an inability of reducing a manufacturing cost. Accordingly, they are not able to be utilized generally, which is very imperfect.

### SUMMARY OF THE INVENTION

The present invention is to provide a retractable safety syringe, in order to facilitate retracting a needle after taking an injection, and to enable an easier manufacturing for reducing an imperfection rate and a cost.

The aforementioned syringe comprises a barrel, a seat, and a push rod, wherein a tubular exit is located at an end of the barrel, a bore is located at an inner rim of a bottom of the exit, a thin breakage part surrounds a periphery of the end, and a bolt is located at a top end of the push rod. After pushing the push rod into the barrel for accomplishing injecting, the bolt of push rod is locked into the bore at the inner rim of the barrel exit to retract the barrel, such that the breakage part at the end of barrel is broken by a stretching force, and the needle is retracted into the barrel.

The bore located at the inner rim of bottom end of barrel exit in the aforementioned syringe structure is a sheet whose center is provided with a threaded opening, and a slant stage member is located at a bottom edge of the bolt of push rod, such that the bolt can be properly rotated after entering the opening of the sheet, so as to be locked on the sheet, for facilitating a retraction of the push rod. Furthermore, as the needle being retracted is in a slant condition by the stage member, the syringe can be prevented from being recycled and causing contamination.

In the aforementioned syringe, the breakage part located in the barrel is a groove in a continuous point shape.

In the aforementioned syringe, a ring of groove is located on a surface of the barrel at a position close to an end, such that a cover having a through-hole in a center and a latching hooks at sides can be covered on the end and latched into the groove with the latching hooks, thereby forming a restrain to the end for preventing damage to the breakage part of the barrel upon taking an injection.

To enable a further understanding of the said objectives and the technological methods of the invention herein, the brief description of the drawings below is followed by the detailed description of the preferred embodiments.

### BRIEF DESCRIPTION OF THE DRAWINGS

FIG. 1 shows a cutaway view of a conventional syringe.

FIG. 2 shows a cutaway view of another conventional syringe.

FIG. 3 shows a cutaway view of a third conventional syringe.

FIG. 4 shows a perspective view of the present invention.

FIG. 5 shows a perspective view of compartments of the present invention.

FIG. 6 shows a cutaway view of the present invention.

FIG. 6A shows a blowup view of part A of FIG. 6.

FIG. 7 shows a schematic view of an implementation of taking an injection with the present invention.

FIG. 8 shows a schematic view of an implementation of locking a bolt onto a bore of the present invention.

FIG. 9 shows another schematic view of an implementation of locking a bolt onto a bore of the present invention.

FIG. 10 shows a third schematic view of an implementation of locking a bolt onto a bore of the present invention.

FIG. 11 shows a schematic view of an implementation of pulling back of the present invention.

FIG. 12 shows a schematic view of an implementation of breaking a push rod of the present invention.

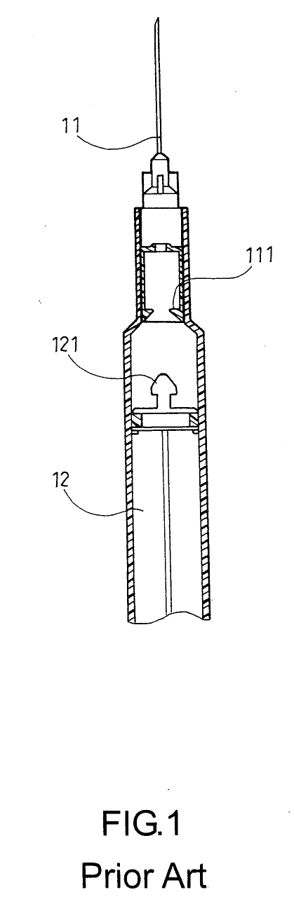

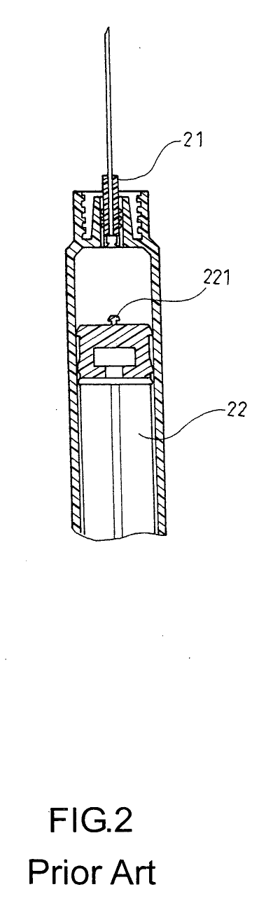

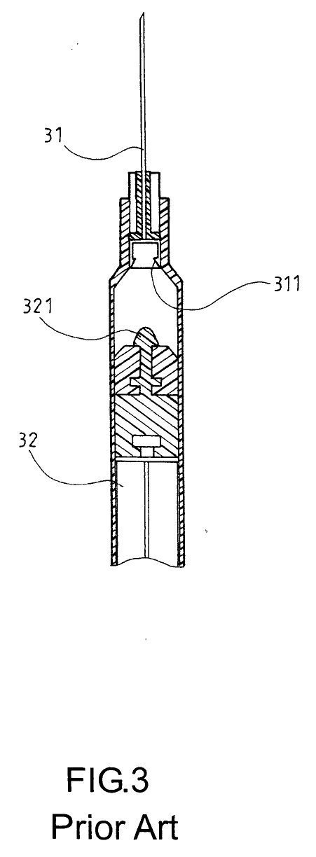

### DETAILED DESCRIPTION OF THE PREFERRED EMBODIMENTS

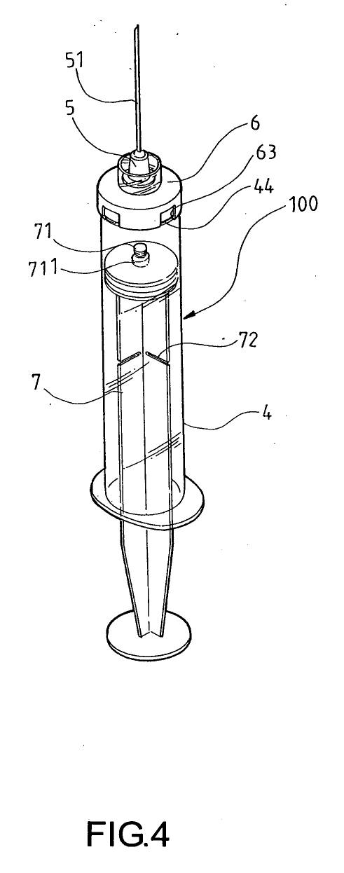

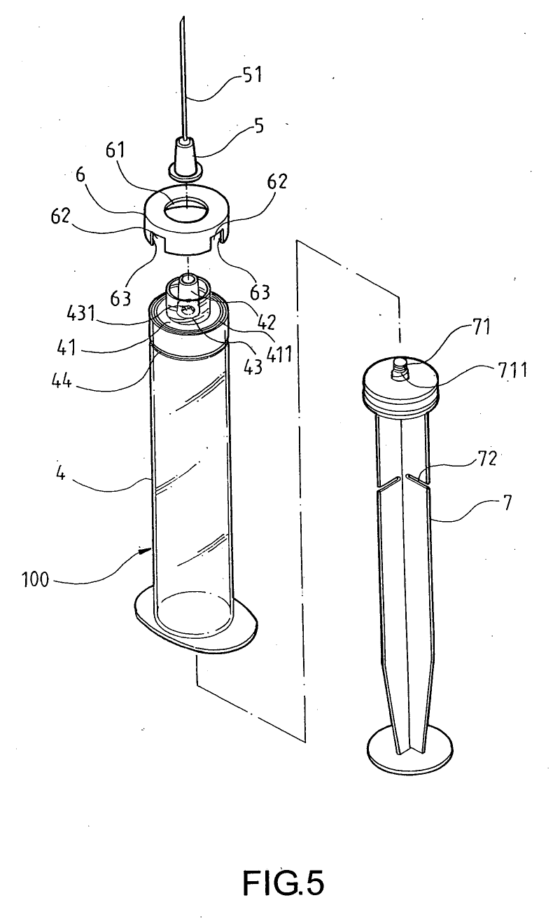

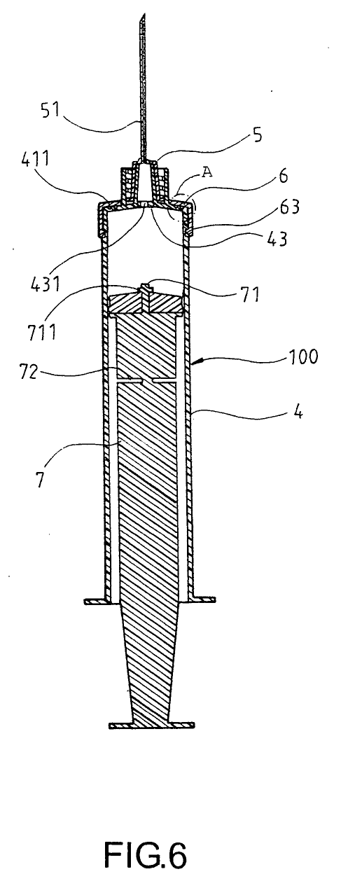

Referring to FIG. 4, FIG. 5, FIG. 6 and FIG. 6A, the present invention comprises primarily a barrel 4, a seat 5, a cover 6, and a push rod 7, wherein a tubular exit 42 is located at an end 41 of the barrel 4 for fixing the seat 5 having a needle 51, a sheet 43 is located at an inner rim of bottom of the exit 42, a circular bore 431 is located at a center of the sheet 43, a thin breakage part constituted by a groove in a continuous point shape is located at a periphery of the end 41 of the barrel 4, and a ring of groove 44 is located on a surface of the barrel 4 close to the end 41.

The cover 6 is in a shape of cap whose center is provided with a through-hole 61, whose sides are provided with notches 62, and whose inner rim at bottom of sides is provided with latching hooks 63, such that when the cover 6 is covered on the end 41 of the barrel 41, the latching hooks 63 at the bottom edge can be latched into the groove 44 on the barrel surface, and the seat 5 and the needle 51 can be fixed on the exit 42 of the barrel 4.

A rod-shape bolt 71 is located at a center of a top end of the push rod 7, a slant stage member 711 with a larger diameter is located at a bottom edge of the bolt 71, and long notches 72 are located at front edges of stems of the push rod 7.

Accordingly, a retractable safety syringe 100 is constituted by assembling the aforementioned compartments. Upon using the present invention, a medicine in the barrel 4 is injected into a patient's body by the push rod 7, and the bolt 71 of the push rod 7 is locked into the bore 431 of the barrel 4 after accomplishing the injection. The groove 411 of the breakage part of the barrel 4 is broken by a stretching force upon retracting the push rod 7, thereby retracting the exit 42, the seat 5, and the needle 51 into the barrel 4, for preventing the used syringe 100 from being recycled, and achieving a purpose of avoiding contamination of the needle 51.

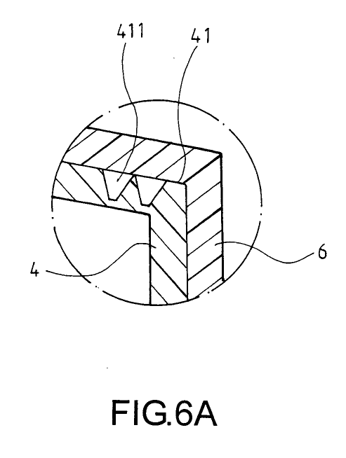

Referring to FIG. 7, upon using the present invention, a medicine 8 is filled into the barrel 4 by retracting the push rod 7, and then injected into a patient's body by pushing forward the push rod 7 during taking an injection.

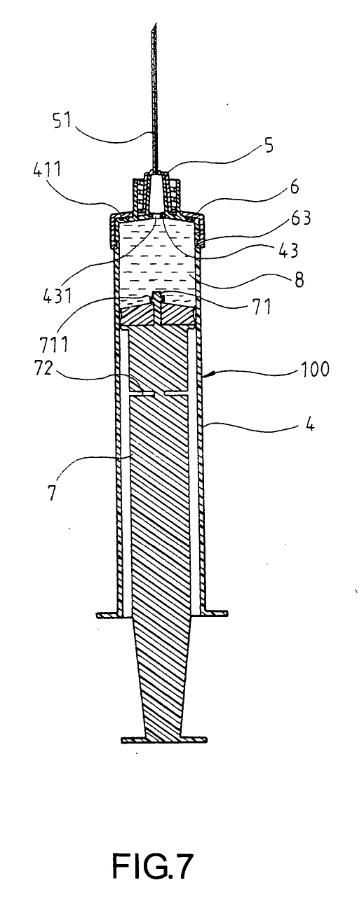

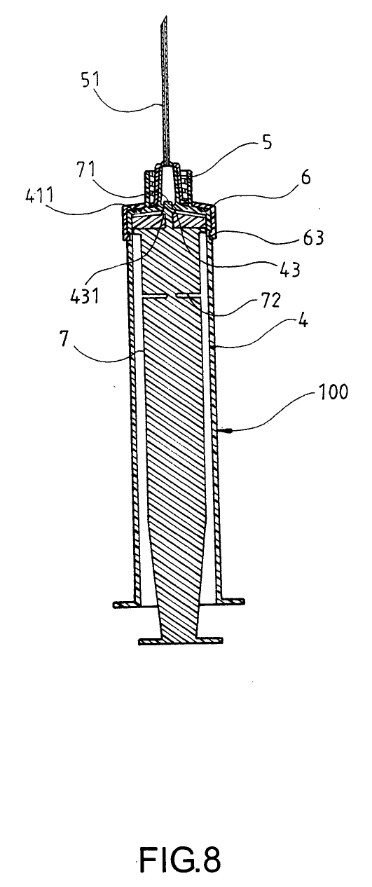

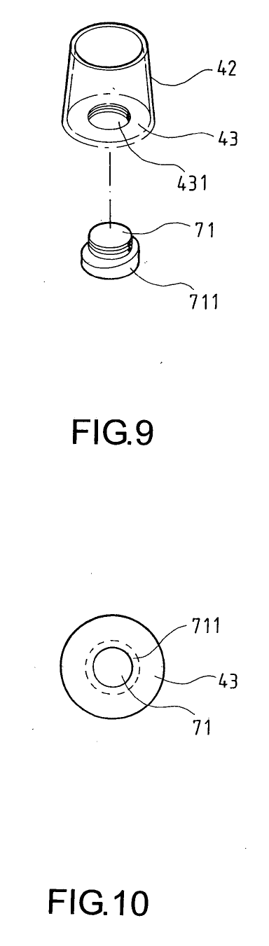

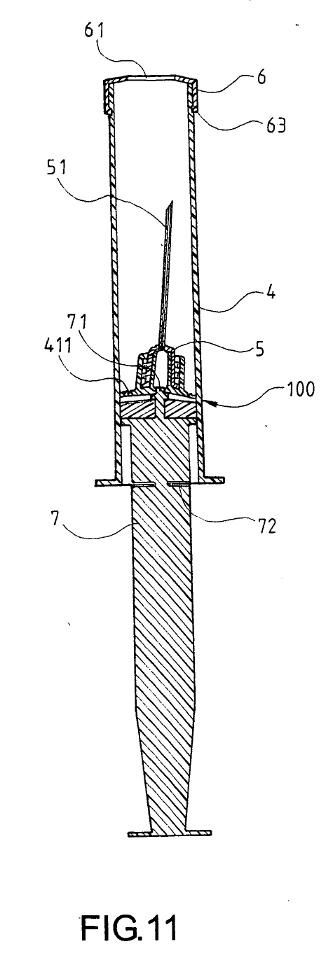

Referring to FIG. 8, FIG. 9, and FIG. 10, when the injection is accomplished, the push rod 7 is pushed forward again, so as to push the bolt 7 into the bore 431 of the sheet 43 of the barrel 4. After entering the bore 431, the bolt 71 is locked onto the sheet 43 by properly rotating the push rod 7. In order to more smoothly rotate the push rod 7, the bore 431 of the sheet 43 is provided with threads which are larger than those of the bolt 71, and the push rod 7 can be retracted after locking the bolt 71. In addition, as the bottom edge of the bolt 71 is provided with the slant stage member 711, the seat 5 and the needle 51 are in a slant position, thereby preventing the needle 51 from being pushed back. Referring to FIG. 11, when retracting the push rod 7, as the bolt 71 is locked onto the sheet 43 of the barrel 4, the point-shape groove 411 at the periphery of the end 41 is broken by a stretching force, thereby retracting the seat 5 and the broken end 41 into the barrel 4, for avoiding reuse and contamination of the needle 5.

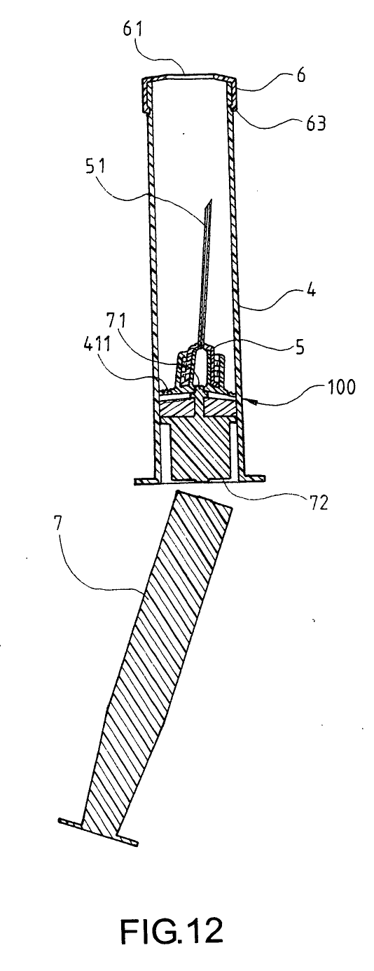

Referring to FIG. 12, after the push rod 7 is retracted to a certain length, it can be broken along a rim of opening of the long notches 72, such that the syringe 100 is totally unusable.

During a process of taking an injection with the present invention, the cover 6 is stopped by the end 41 of the barrel 4; therefore, damage to the groove 411 of the breakage part by an injection pressure can be avoided.

Accordingly, the present invention uses a barrel with a breakage part and a push rod with a bolt to retract a needle into the barrel after using a syringe, thereby preventing the syringe from being recycled and reducing a manufacturing cost and imperfection rate of the retractable safety syringe.

It is of course to be understood that the embodiments described herein is merely illustrative of the principles of the invention and that a wide variety of modifications thereto may be effected by persons skilled in the art without departing from the spirit and scope of the invention as set forth in the following claims.

What is claimed is:

1. A retractable safety syringe comprising a barrel comprising a tubular exit for fixing a seat is located at an end, a bore is located at an inner rim of bottom of the exit, a thin breakage part is located at a periphery of the end, and a ring of groove is located on a surface close to the end; a cap-shape cover being covered on the end of barrel, with a through-hole in a center of the cover allowing for the seat to pass in, notches at sides, and latching hooks at an inner rim of sides for latching into the groove of barrel; a push rod located inside the barrel, with a bolt at a center position in a front end; by assembling the aforementioned compartments, the bolt of push rod being locked into the bore of barrel after taking an injection, so as to break the breakage part from a stretching force upon retracting the push rod, thereby retracting the seat and needle into the barrel; the cover being able to prevent damage to the groove of breakage part from an injection pressure.

2. The retractable safety syringe according to claim 1, comprising the bore at the inner rim of bottom of exit of barrel is provided with a sheet whose center position is provided with a threaded opening.

3. The retractable safety syringe according to claim 1, comprising the breakage part at the periphery of end of barrel is a continuous groove which is easily broken.

4. The retractable safety syringe according to claim 1, comprising a slant stage member is located at a bottom edge of the bolt in a front end of the push rod, so as to cause the seat and the needle to be in a slant position upon retracting, thereby preventing the syringe from being recycled or causing an infection.
